(19) United States
(12) Patent Application Publication
(10) Pub. No.: US 2025/0177604 A1
(43) Pub. Date: Jun. 5, 2025

(54) MEDICATED SKIN CONCEALER

(71) Applicant: Maaike Snoep, Jersey City, NJ (US)

(72) Inventor: Maaike Snoep, Jersey City, NJ (US)

(21) Appl. No.: 18/965,568

(22) Filed: Dec. 2, 2024

Related U.S. Application Data
(60) Provisional application No. 63/629,776, filed on Nov. 30, 2023.

(51) Int. Cl. A61L 26/00

(52) U.S. Cl. CPC A61L 26/0066; A61L 26/0004; A61L 26/0019; A61L 26/0057; A61L 2300/406; A61L 2430/34

(57) ABSTRACT

The present disclosure relates to medicated skin concealers and methods associated therewith.

### BACKGROUND

### 1. Technical Field

The present disclosure relates to a medicated skin concealer, and more particularly, to a skin concealer including one or more medicaments suitable for covering a wound and/or as a first aid treatment. Methods of making and using a medicated skin concealer are also provided.

### 2. Related Art

Medicated topical ointments or sprays, such as Neosporin®, are known. The ointments are petroleum based and can be viscous or goopy, as well as highly irritating, particularly along the face around the eyes, nose, or mouth. The sprays forms of these products are designed to be easier to apply to an area of the skin, but due to the liquid nature of the spray product and the application of gravity thereto, these products can immediately begin to run away from the area of application. These medicated ointments or sprays also do not hide the area of application from plain sight. And these medicated ointments or sprays are clearly noticeable and different from a patient's natural skin tone. As such, these sorts of medicated ointments or sprays are not designed to conceal.

In some instances, a liquid topical product such as Liquid Skin® may include an adhesive or glue base, such as a cyanoacrylate base, to adhere the liquid product directly to the skin. However, the product is designed to adhere to the skin and protect the skin for 3-5 days. The liquid topical glues do not conceal and are designed for a single application which stays on the skin for an extended period of time or days, unlike some cosmetic products.

It would be beneficial to provide a medicated skin concealer designed to deliver an effective amount of the concealer to cover and/or hide a part of a skin from plain sight and which generally matches a natural skin tone of a patient, may be non-irritating, and can be reapplied more than once daily, without running away from the site of application or adhering directly to the skin like a glue.

### SUMMARY

The present disclosure provides medicated skin concealers designed for covering, hiding, and/or treating a wound in the skin. The medicated skin concealers provided herein are designed to be applied to a wound in a manner which hides the wound from plain sight and generally matches a natural skin tone of the skin the concealer is applied thereto so the medicated concealer is also not easily visible in plain sight. The medicated skin concealers provided herein are designed to be applied at least once daily, if not more than once daily. The medicated skin concealers provided herein are designed to not run-away from the site of application without adhering (i.e., forming a chemical bond) with the tissue. The medicated skin concealers provided herein may also be designed as non-irritating, particularly to the eyes, nose, and/or mouth along the face.

The medicated concealers include at least a concealer composition and a medicament component. The medicament component may include at least one of bacitracin, neomycin, polymyxin, and/or pramoxine. As provided throughout the present disclosure, any recitation of bacitracin, neomycin, polymyxin, or pramoxine is intended to encompass any or all forms or salts of the such medicaments. For example, without limitation, the medicament component may include at least one of bacitracin zinc, neomycin sulphate, polymyxin sulphate, and/or pramoxine hydrochloride.

In some embodiments, the medicated skin concealer is a hydrous concealer.

In some embodiments, the medicated skin concealer is an emulsion including at least an aqueous component, an oil component, and a pigment component. One or more additive components may be further included.

In some embodiments, the medicated skin concealer includes an aqueous component which is predominantly water.

In some embodiments, the medicated skin concealer includes an oil component which is predominantly one or more polyorganosiloxanes.

In some embodiments, the medicated skin concealer includes a pigment component which is predominantly titanium dioxide.

In some embodiments, the medicated skin concealer includes at least one additive component including a polyorganosiloxane, a PEG/PPG compound, or a clay such as bentonite.

Methods of manufacturing and using the various medicated skin concealers are also provided.

### DETAILED DESCRIPTION

Embodiments of the presently disclosed medicated skin concealer, and the methods of manufacturing or methods of treating associated therewith, will now be described in detail.

As used herein, all ranges provided are intended to be inclusive of the recited range and may include any combination of sub-ranges falling between the recited range. For example, a range of about 1 to about 10 is inclusive to include 1 and 10 and may include a sub-range of 1-2, 1-3, 4-7, 9-10, etc.

As used herein, the term “about” is intended to be within 10% of the recited number. For example, “about 10” may include from 9 to 11.

As used herein, “substantially free” represents having less than 0.05% by weight of a particular ingredient or component.

All percentages of ingredients or components provided herein are based on weight of the ingredient or component with respect to the overall weight of the component or composition.

The medicated skin concealer is designed to topically cover and/or treat a wound in a skin of a patient. The concealer includes a concealer composition and a medicament component. The medicament component includes one or more medicaments designed to be applied topically to a wound one or more times a day. The wound may be an open wound (e.g., a wet wound such as a scrape, a burn, a cut, etc.) or a closed wound (e.g., a dry wound with a scab formed thereon, or an closed incision). An open wound generally is a wound which may include blood or exudate. A closed wound generally may be dry or scabbed over or sutured closed.

The concealer and/or medicament component may include any suitable topical wound-treating medicament. Some non-limiting examples of suitable wound-treating (and/or first-aid) medicaments include: antibiotics (e.g., bacitracin, polymyxin, neomycin, mupirocin, gentamicin, erythromycin, cefazolin, clarithromycin, cephalexin, cefadroxil, etc.), antivirals (e.g., acyclovir, famciclovir, penciclovir, nirmatrelvir, emtricitabine, tenofovir, ritonavir, abacavir, etc.), pain-relievers (e.g., acetaminophen, codeine, morphine, oxycodone, etc.), anti-inflammatory agents (e.g., aspirin, ibuprofen, naproxen, diclofenac, celecoxib, meloxicam, etc.), anesthetics (e.g., lidocaine, benzocaine, bupivacaine, etc.), clotting agents (e.g., alum, clotting factors, etc.), steroids (e.g., hydrocortisone, clobetasone, mometasone, beclomethasone, prednisone, fluticasone, etc.) anti-itch agents (e.g., diphenhydramine, pramoxine, etc.), skin protectants (e.g., calamine, zinc oxide, aluminum acetate, etc.), individually or in any combination. The medicament component may be free of any additional non-medicament ingredients.

In some embodiments, the concealer includes a concealer composition and a medicament component which includes one or more medicaments selected from bacitracin, polymyxin, neomycin, or pramoxine, individually or in any combination. For example, the concealer may have a medicament component including bacitracin, polymyxin, neomycin, and pramoxine. In another example, the concealer may have a medicament component including only one of bacitracin, polymyxin, neomycin, or pramoxine.

In some embodiments, the medicated concealer includes from about 0.01 to about 10% bacitracin. In some embodiments, the medicated concealer includes from about 0.1 to about 2% bacitracin.

In some embodiments, the medicated concealer includes from about 0.01 to about 10% neomycin. In some embodiments, the medicated concealer includes from about 0.1 to about 2% neomycin.

In some embodiments, the medicated concealer includes from about 0.01 to about 10% polymyxin. In some embodiments, the medicated concealer includes from about 0.1 to about 2% polymyxin.

In some embodiments, the medicated concealer includes from about 0.01 to about 10% pramoxine. In some embodiments, the medicated concealer includes from about 0.1 to about 2% pramoxine.

In some embodiments, the medicated concealer includes from about 0.01 to about 10% of a medicament component including bacitracin, polymyxin, neomycin, and pramoxine. In some embodiments, the medicated concealer includes from about 0.1 to about 2% of a medicament component including bacitracin, polymyxin, neomycin, and pramoxine.

The medicament component may be combined with any concealer composition to form a skin concealer suitable for delivering the medicament to the wound while hiding the wound from plain sight. The medicated concealer is configured to display a texture and/or softness suitable for application directly to a wound without clumping, falling apart, and/or running away from the wound, regardless if the wound is open or closed.

The concealer is designed generally to match the skin-tone of the patient so as to blend into the skin of the patient thereby hiding the wound from plain sight. The medicated skin concealer is also designed to provide an effective amount of a medicament to treat or aid in healing of the wound and/or to treat or aid in reducing a symptom associated with the wound (e.g., swelling, pain, itchiness, discoloration, and the like).

The medicated skin concealer may be designed to be applied to the epidermal layer of the skin without being absorbed into the deeper layer of the skin so as not to disappear from the outer layer of the skin thereby maintaining the ability to cover a wound from plain sight.

The medicated skin concealer may further be designed to not adhere opposing sides of a wound to each other in a closed configuration.

The medicated skin concealer may further be designed to not form a chemical bond with the wound and/or the skin immediately surrounding the wound.

In some embodiments, the skin concealer is a hydrous skin concealer including a medicament component and a hydrous concealer composition. The hydrous concealer composition may be an emulsion including at least an aqueous component (and/or phase), an oil component (and/or phase), a pigment component, and one or more additive components.

The aqueous component may include a predominant amount of water and a minor amount of a combination of an alkylene glycol and a metal silicate. The water may be any type of water including but not limited to one or more of distilled water, sterile water, spring water, tap water, pyrogen-free water, etc. A few non-limiting examples of a suitable alkylene glycol include propylene glycol or ethylene glycol, and a non-limiting example of a suitable metal silicate includes magnesium aluminum silicate.

In some embodiments, the aqueous component of a hydrous concealer composition includes a predominant amount of distilled water and a minor amount of a combination of propylene glycol and magnesium aluminum silicate.

In some embodiments, the water represents about 75-95% of the aqueous component and the combination of the alkylene glycol and the metal silicate represent about 5-25% of the aqueous component. The metal silicate may represent less than 1% of the aqueous component.

In some embodiments, the water represents about 85-90% of the aqueous component and the combination of the alkylene glycol and the metal silicate represent about 10-15% of the aqueous component. The metal silicate may represent less than 0.5% of the aqueous component.

In some embodiments, the water represents about 75-95% of the aqueous component and the combination of the propylene glycol and magnesium aluminum silicate represent about 5-25% of the aqueous component. The magnesium aluminum silicate may represent less than 1% of the aqueous component.

In some embodiments, the water represents about 85-90% of the aqueous component and the combination of the propylene glycol and the magnesium aluminum silicate represent about 10-15% of the aqueous component. The magnesium aluminum silicate may represent less than 0.5% of the aqueous component.

The oil component of the concealer may include a predominant amount of one or more polyorganosiloxanes and a minor amount of a combination of an oil, a wax, and pigment component. Some non-limiting examples of suitable polyorganosiloxanes include linear siloxanes (e.g., dimethicone, hexamethyldisiloxane, octamethyltrisiloxane, decamethyltetrasiloxane, dodecamethylpentasiloxane, etc.), cyclic siloxanes (e.g., cyclomethicone, octamethylcyclotetrasiloxane, decamethylcyclopentasiloxane, dodecamethylcyclohexasiloxane, etc.), alkyl siloxanes (e.g., C₂₀₋₂₄ alkyl dimethicone, C₂₄₋₂₈-alkyl dimethicone, C₃₀₋₄₅ alkyl dimethicone, etc.), and the like. A few non-limiting examples of a suitable wax includes carnauba wax, beeswax, paraffin wax, lanolin wax, candelilla wax, and the like. A few non-limiting examples of a suitable oil includes mineral oil, castor oil, vegetable oil, coconut oil, soybean oil, sunflower oil, avocado oil, olive oil, and the like.

The pigment component may include any suitable colorant and/or pigment. Some non-limiting examples include titanium dioxide, zinc oxide, red iron oxide, yellow iron oxide, black iron oxide, and the like. In some embodiments, the pigment component includes a predominant amount of titanium dioxide mixed with a minor amount of one or more of red iron oxide, yellow iron oxide, or black iron oxide.

In some embodiments, the oil component of a hydrous concealer composition includes a predominant amount of a combination of dimethicone, cyclomethicone, and C₃₀₋₄₅ alkyl dimethicone with a minor amount of a combination of mineral oil, carnuba wax, and a pigment component.

In some embodiments, the oil component of a hydrous concealer composition includes a predominant amount of a combination of dimethicone, cyclomethicone, and C₃₀₋₄₅ alkyl dimethicone with a minor amount of a combination of mineral oil, carnuba wax, titanium dioxide, red iron oxide, yellow iron oxide, and black iron oxide.

In some embodiments, the plurality of different polyorganosiloxanes represent about 55-90% of the oil component and the combination of the oil, the wax, and the pigment component represent about 10-45% of the oil component.

In some embodiments, the plurality of different polyorganosiloxanes represent about 60-75% of the oil component and the combination of the oil, the wax, and the pigment component represent about 25-40% of the oil component.

In some embodiments, the combination of the dimethicone, cyclomethicone, and C₃₀₋₄₅ alkyl dimethicone represent about 55-90% of the oil component and the combination of the mineral oil, the carnauba wax, the titanium dioxide, the red iron oxide, the yellow iron oxide, and the black iron oxide represent about 10-45% of the oil component.

In some embodiments, the combination of the dimethicone, cyclomethicone, and C₃₀₋₄₅ alkyl dimethicone represent about 60-75% of the oil component and the combination of the mineral oil, the carnauba wax, the titanium dioxide, the red iron oxide, the yellow iron oxide, and the black iron oxide represent about 25-40% of the oil component.

The one or more additive components may include a first additive component configured to be combined with the emulsion prior to the addition of any medicament component and a second additive component configured to be combined with the emulsion after the addition of any medicament component. The first additive component includes one or more of dimethicone or a PEG/PPG (polyethylene glycol/polypropylene glycol) compound. Some non-limiting examples of a PEG/PPG compound include PEG/PPG-18/18 dimethicone, PEG/PPG-19/19 dimethicone, PEG/PPG-20/20 dimethicone, PEG/PPG 20/23 dimethicone, PEG/PPG 20/22 butyl ether dimethicone, PEG/PPG 23/6 dimethicone, PEG/PPG 20/15 dimethicone, and the like.

In some embodiments, the first additive component includes about 40-60% dimethicone and about 40-60% a PEG/PPG compound.

In some embodiments, the first additive component includes about 40-60% dimethicone and about 40-60% PEG/PPG 18/18 dimethicone.

In some embodiments, the first additive component includes only equal parts of

both dimethicone and a PEG/PPG compound.

In some embodiments, the first additive component includes only equal parts of both dimethicone and a PEG/PPG 18/18 dimethicone.

The second additive component includes at least one clay or organically modified clay. The clay or organically modified clay may be selected from some non-limiting examples such as bentonite, kaolinite, smectite, montmorillonite, hectorites, saponites, and the like.

In some embodiments, the second additive component includes at least bentonite.

In some embodiments, the second additive component includes only bentonite.

In some embodiments, the medicated concealer includes a first additive component including equal parts of both dimethicone and a PEG/PPG 18/18 dimethicone and a second additive including only bentonite.

The skin concealer may further include one or more optional ingredients. Some non-limiting examples of suitable optional ingredients include preservatives, fragrances, vitamins, minerals, SPF ingredients, antioxidants, etc. Any optional ingredient represents less than 5% of the concealer. In some embodiments, any optional ingredient represents less than 1% of the concealer.

The skin concealer may be substantially free of some materials. For example, the skin concealer may be free of any topical adhesive or glue materials, such as cyanoacrylates. In another example, the skin concealer may be substantially free of petrolatum.

In some embodiments, the aqueous component represents about 25-45% of the medicated skin concealer, the oil component represents about 25-45% of the medicated skin concealer, the pigment component represents about 0.1-12% of the medicated skin concealer, the first additive component represents about 15-35% of the medicated skin concealer, the medicament component represents about 0.1-12% of the medicated skin concealer, and the second additive component represents about 0.1-12% of the medicated skin concealer.

In some embodiments, the aqueous component represents about 30-35% of the medicated skin concealer, the oil component represents about 30-35% of the medicated skin concealer, the pigment component represents about 5-10% of the medicated skin concealer, the first additive component represents about 20-30% of the medicated skin concealer, the medicament component represents about 1-6% of the medicated skin concealer, and the second additive component represents about 1-4% of the medicated skin concealer.

In some embodiments, the medicated skin concealer includes a hydrous concealer composition with a medicament component including one or more of bacitracin, neomycin, polymyxin, and/or pramoxine, wherein the aqueous component represents about 25-45% of the medicated skin concealer, the oil component represents about 25-45% of the medicated skin concealer, the pigment component represents about 0.1-12% of the medicated skin concealer, the first additive component represents about 15-35% of the medicated skin concealer, the medicament component represents about 0.1-12% of the medicated skin concealer, and the second additive component represents about 0.1-12% of the medicated skin concealer. Each of the aqueous component, the oil component, the pigment component, and the first and second additives components being any as provided in the various embodiments herein.

In some embodiments, the medicated skin concealer includes a hydrous concealer composition with a medicament component including one or more of bacitracin, neomycin, polymyxin, and/or pramoxine, wherein the aqueous component represents about 30-35% of the medicated skin concealer, the oil component represents about 30-35% of the medicated skin concealer, the pigment component represents about 5-10% of the medicated skin concealer, the first additive component represents about 20-30% of the medicated skin concealer, the medicament component represents about 1-6% of the medicated skin concealer, and the second additive component represents about 1-4% of the medicated skin concealer. Each of the aqueous component, the oil component, the pigment component, and the first and second additives components being any as provided in the various embodiments herein.

The present disclosure further provides methods of manufacturing and/or forming a medicated skin concealer. The methods generally include combining a concealer composition, and particularly a hydrous concealer composition, with a medicament component to form the medicated concealer. The medicament component may include one or more of bacitracin, neomycin, polymyxin, and/or pramoxine.

In some embodiments, the method of manufacturing and/or forming a medicated skin concealer includes one or more of the following steps: preparing an aqueous component including a predominant amount of water and a minor amount of magnesium aluminum silicate and an alkylene glycol, such as propylene glycol, at a first temperature; preparing an oil component including a predominant amount of a plurality of different polyorganosiloxanes, such as dimethicone, cyclomethicone, and C30-45 alkyl dimethicone, and a minor amount of an oil, such as mineral oil, and a wax, such as carnauba wax, at a second temperature; adding a pigment component to the oil component, the pigment component including a plurality of pigment ingredients, particularly two or more of titanium dioxide, red iron oxide, yellow iron oxide, and/or black iron oxide, at a third temperature; combining the aqueous component and the pigment-containing oil component to form an emulsion at a fourth temperature; adding a first additive component to the emulsion, the first additive component including dimethicone and a PEG/PPG compound, such as PEG/PPG 18/18, at a fifth temperature; adding a medicament component, particularly including at least one of the following: bacitracin, neomycin, polymyxin, and/or pramoxine, to the emulsion at a sixth temperature to form a medicated emulsion; and adding a second additive component including a clay, particularly bentonite, to the medicated emulsion at a seventh temperature. The medicated emulsion may be further mixed and homogenized to a smooth, lump-free, anti-clumping skin concealer at room temperature.

The first, second, third, and/or fourth temperatures are independently greater than 50° C. In some embodiments, the first, second, third, and/or fourth temperatures independently range between about 60° C. to about 90° C. In some embodiments, the first, second, third, and/or fourth temperatures independently range between about 70° C. to about 80° C.

The fifth, sixth, and seventh temperatures are lower than the first, second, third, and fourth temperatures. In some embodiments, the seventh temperature is less than the sixth temperature which is less than the fifth temperature.

In some embodiments, the fifth temperature ranges from about 40° C. to about 45° C., the sixth temperature ranges from about 35° C. to about 40° C. and is lower than the fifth temperature, and the seventh temperature ranges from about 25° C. to about 30° C. and is lower than the sixth temperature.

The present disclosure further provides methods of using a medicated skin concealer as provided herein. The methods generally include applying the medicated skin concealer directly over a wound to cover and/or hide the wound from sight, without the need to apply a bandage or covering; and reapplying the medicated skin concealer one or more times per day. The amount of medicated skin concealer applied may vary depending upon the size of the wound, but is generally the amount sufficient to completely cover the surface area of the wound including any immediately surrounding tissue to allow the skin concealer to blend in with the patient's natural skin tone.

In some embodiments, the skin concealer is applied over an open wound, such as a scratch, cut, abrasion, rash, etc. In some embodiments, the skin concealer is applied over a closed wound.

Reapplication of the skin concealer multiple times per day may or may not be necessary to conceal the wound, however it may be necessary and/or encouraged to continue to deliver the medicament to the wound and/or skin. In some embodiments, the methods of using the skin concealer may further include the step of removing the concealer after a period of time to reapply a new effective amount of medicated skin concealer. Alternatively, in some embodiments, the methods of using the skin concealer may further include reapplying the concealer directly over prior applications of the concealer, in whole or in part. It is envisioned that the concealer may be organically removed from the wound, for example, by degradation of the concealer, absorption of the concealer, exposure of the concealer to wound exudate or perspiration, physically removed via contact with another object, such as clothing.

In some embodiments, the medicated concealer is a hydrating medicated concealer wherein the medicated concealers provided herein include a combination of base materials in the water and/or oil components which cover the wound and lock any moisture in the wound thereby hydrating the wound from inside without the use of petroleum and/or any other skin irritating material.

In some embodiments, the medicated skin concealer may be applied multiple times per day. For example, the concealer may be applied up to 12 times per day. The medicated skin concealer may be applied every 2 hours, or every 4 hours, or every 6 hours, or every 8 hours, or every 12 hours.

In some embodiments, each gram of the medicated concealer includes: about 250-about 1000 IU, particularly 500 UI, of bacitracin; about 5000 UI-about 15000 UI, particularly 10000 IU, of polymyxin; about 1 to about 10 mg, particularly 3.5 mg, of neomycin; and/or about 5 to about 20 mg, particularly 10 mg, of pramoxine,

In some embodiments, about 0.1 to about 5 grams of the medicated skin concealer, and particularly a medicated skin concealer including one or more of bacitracin, polymyxin, neomycin, and/or pramoxine, is applied to a wound per application. In some embodiments, about 0.25 to about 2.5 grams of the medicated skin concealer is applied to a wound per application. In some embodiments, about 0.5 to about 1.5 grams of the medicated skin concealer is applied to a wound per application.

In some embodiments, the amount of the medicated skin concealer, and particularly a medicated skin concealer including any of the concealer compositions provided herein and a medicament component having one or more of bacitracin, polymyxin, neomycin, and/or pramoxine, applied to a wound is about 0.1 to about 5 grams/cm² of surface area of the wound and any immediately surrounding tissue per application.

In some embodiments, the amount of the medicated skin concealer, and particularly a medicated skin concealer including any of the concealer compositions provided herein and a medicament component having one or more of bacitracin, polymyxin, neomycin, and/or pramoxine, applied to a wound is about 0.25 to about 2.5 grams/cm² of surface area of the wound and any immediately surrounding tissue per application.

In some embodiments, the amount of the medicated skin concealer, and particularly a medicated skin concealer including any of the concealer compositions provided herein and a medicament component having one or more of bacitracin, polymyxin, neomycin, and/or pramoxine, applied to a wound is about 0.5 to about 1.5 grams/cm² of surface area of the wound and any immediately surrounding tissue per application.

In some embodiments, the medicated skin concealer, and particularly a medicated skin concealer including any of the concealer compositions provided herein and a medicament component having one or more of bacitracin, polymyxin, neomycin, and/or pramoxine, is applied to a wound every 2-12 hours, and particularly every 3-6 hours, in an amount of about 0.1 to about 5 grams/cm² of surface area of the wound and any immediately surrounding tissue per application.

In some embodiments, the medicated skin concealer, and particularly a medicated skin concealer including any of the concealer compositions provided herein and a medicament component having one or more of bacitracin, polymyxin, neomycin, and/or pramoxine, is applied to a wound every 2-12 hours, and particularly every 3-6 hours, in an amount of about 0.25 to about 2.5 grams/cm² of surface area of the wound and any immediately surrounding tissue per application.

In some embodiments, the medicated skin concealer, and particularly a medicated skin concealer including any of the concealer compositions provided herein and a medicament component having one or more of bacitracin, polymyxin, neomycin, and/or pramoxine, is applied to a wound every 2-12 hours, and particularly every 3-6 hours, in an amount of about 0.5 to about 1 gram/cm² of surface area of the wound and any immediately surrounding tissue per application.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplification of embodiments. Those skilled in the art will envision other modifications within the scope and spirit of the claims appended hereto.

### Example 1

A medicated skin concealer for use with covering a wound in a patient's skin was formed as provided hereinbelow. The concealer included a concealer composition including an aqueous phase or component, an oil phase or component, a pigment component, a first additive component, a second additive component, and a medicament component.

### Preparation of Aqueous Phase:

An aqueous phase was formed by combining the following ingredients in the following amounts:

| Ingredient | Amount |
|---|---|
| Distilled Water | 278.2 mg |
| Magnesium Aluminum Silicate | 0.75 mg |
| Propylene Glycol | 37.59 mg |

The distilled water was weighted and poured into a clean stainless steel mixing vessel. Magnesium aluminum silicate was slowly added into the water while stirring continuously to avoid lumps. The mixture was heated to approximately 70° C. to fully hydrate the magnesium aluminum silicate. The propylene glycol was added and the stirring continued to ensure complete dissolution to form the aqueous phase or component.

### Preparation of Oil Phase:

An oil phase was formed separately by combining the following ingredients in the

following amounts:

| Ingredient | Amount |
|---|---|
| Cyclomethicone | 127.82 mg |
| Dimethicone | 90.23 mg |
| C30-45 alkyl dimethicone | 37.59 mg |
| Light Mineral oil | 37.59 mg |
| Carnuba wax | 8.55 mg |

The cyclomethicone, dimethicone, C30-45 alkyl dimethicone, light mineral oil, and carnuba wax were each weighed and mixed together in a separate mixing vessel to form an oil phase mixture. The oil phase mixture was heated to between 70° C. and 80° C. to melt the Carnuba wax and mixed thoroughly using a high-shear mixer to form the oil phase or component.

### Pigment Dispersion:

A pigment dispersion was formed in yet another container by combining the following pigment ingredients in the following amounts in a small amount of the oil phase.

| Ingredient | Amount |
|---|---|
| Titanium dioxide | 75.19 mg |
| Iron oxide (yellow | 11.28 mg |
| Iron Oxide (red) | 2.26 mg |
| Iron Oxide (black) | 0.75 mg |

A high-shear mixer or homogenizer was used to ensure the pigment ingredients were uniformly distributed in the oil phase.

### Combining of Phases:

The aqueous phase was slowly added to the oil phase (with pigment ingredients)

while the temperature was maintained at 70° C.-80° C. and stirring continuously with a homogenizer to form a smooth, uniform emulsion.

### Addition of First Additive Component

A first additive component was formed by combining the following additive ingredients in the following amounts:

| Ingredient | Amount |
|---|---|
| Dimethicone | 127.82 mg |
| PEG/PPG 18/18 dimethicone | 127.82 mg |

The emulsion was cooled while continuing to mix. At around 40° C., the first additive component was combined with the cooling emulsion until homogeneously mixed to improve the texture and consistency of the emulsion.

### Addition of Medicament Component

A medicament component was formed by combining the following medicament ingredients in the following amounts:

| Ingredients | Amount | |
|---|---|---|
| Bacitracin Zinc | 6.755 | mg (500 IU) |
| Neomycin sulphate | 3.5 | mg |
| Polymyxin B sulphate | 1.27 | mg (10,000 IU) |
| Pramoxine hydrochloride | 10 | mg |

After the emulsion was cooled further to below 40° C., the medicament component was added to the cooling emulsion including the first additive component. Stirring gently was continued to avoid degrading the medicament ingredients in the emulsion due to the remaining heat.

### Addition of a Second Additive Component:

A second additive component was added to the cooling medicated emulsion. The second additive component included 15.04 mg of Bentonite. The Bentonite was added while continuously mixing to further prevent clumping. The Bentonite was fully dispersed and evenly mixed throughout the medicated emulsion to provide a thicker consistency.

Final Mixing and homogenization:

After all the ingredients and/or components were combined, the medicated mixture was further homogenized to achieve a medicated skin concealer having a smooth, lump-free consistency. The stirring of the medicated mixture was continued until the mixture reached room temperature.

An overall ingredient list of the skin concealer is provided below.

| Phase or | Ingredient | | | |
|---|---|---|---|---|
| Component | No. | Ingredient | Quantity | UOM |
| Medicament | 1 | Bacitracin Zinc | 6.755 | mg |
| | 2 | Neomycin sulphate | 3.5 | mg |
| | 3 | Polymyxin sulphate | 1.27 | mg |
| | 4 | Pramoxine Hydrochloride | 10 | mg |
| Oil | 5 | Cyclomethicone | 127.82 | mg |
| | 6 | Dimethicone | 90.23 | mg |
| | 7 | C30-45 alkyl dimethicone | 37.59 | mg |
| | 8 | Light Mineral Oil | 37.59 | mg |
| | 9 | Carnuba Wax | 8.55 | mg |
| 1^{st} Additive | 10 | Dimethicone | 127.82 | mg |
| | 11 | PEG/PPG 18/18 | 127.82 | mg |
| | | dimethicone | | |
| Aqueous | 12 | Propylene Glycol | 37.59 | mg |
| | 13 | Magnesium Aluminum | 0.75 | mg |
| | | Silicate | | |
| | 14 | Distilled Water | 278.2 | mg |
| 2^{nd} Additive | 15 | Bentonite | 15.04 | mg |
| Pigment | 16 | Titanium Dioxide | 75.19 | mg |
| | 17 | Iron Oxide (Yellow) | 11.28 | mg |
| | 18 | Iron Oxide (Red) | 2.26 | mg |
| | 19 | Iron Oxide (Black) | 0.75 | mg |

What is claimed is:

1. A medicated concealer for covering a wound, the concealer comprising:
a concealer composition and a medicament component, the medicament component including at least one of the following:
bacitracin, neomycin, polymyxin, or pramoxine.

2. The medicated concealer of claim 1, wherein the concealer composition is an emulsion comprising:
an aqueous component, an oil component, a pigment component, a first additive component, and a second additive component.

3. The medicated concealer of claim 2, wherein the aqueous component comprises a predominant amount of water, and a minor amount of an alkylene glycol and magnesium aluminum silicate.

4. The medicated concealer of claim 3, wherein the alkylene glycol is propylene glycol.

5. The medicated concealer of claim 2, wherein the oil component comprises a predominant amount of a plurality of different polyorganosiloxanes and a minor amount of an oil, a wax, and a pigment component.

6. The medicated concealer of claim 5, wherein:
the plurality of different polyorganosiloxanes comprise a linear siloxane, a cyclic methylsiloxane, and an alkyl siloxane;
the oil is mineral oil; and
the wax is carnauba wax.

7. The medicated concealer of claim 6, wherein:
the linear siloxane is dimethicone;
the cyclic methylsiloxane is cyclomethicone; and
the alkyl siloxane is C₃₀₋₄₅ alkyl dimethicone.

8. The medicated concealer of claim 7, wherein the pigment component includes one or more of titanium dioxide, yellow iron oxide, red iron oxide, or black iron oxide.

9. The medicated concealer of claim 5, wherein the first additive component includes a linear siloxane and a PEG/PPG compound.

10. The medicated concealer of claim 9, wherein the linear siloxane is dimethicone and the PEG/PPG compound is PEG/PPG 18/18 dimethicone.

11. The medicated concealer of claim 5, wherein the second additive component is bentonite.

12. The medicated concealer of claim 2, wherein:
the aqueous component includes distilled water, magnesium aluminum silicate, and propylene glycol;
the oil component includes cyclomethicone, dimethicone, C30-45 alkyl dimethicone, mineral oil, Carnuba wax, and a pigment component;
the first additive component includes dimethicone and PEG/PPG 18/18 dimethicone; and
the second additive component includes bentonite.

13. A method of forming a medicated concealer for covering a wound, the method comprising:
combining a hydrous concealer composition and a medicament component to form a homogenized medicated skin concealer, the medicament component including one or more of bacitracin, neomycin, polymyxin, and/or pramoxine.

14. The method of claim 13, wherein the hydrous concealer composition is prepared by:
combining an aqueous component and an oil component to form an emulsion, the aqueous component including water, magnesium aluminum silicate, and an alkylene glycol, and the oil component including a plurality of polyorganosiloxanes, an oil, a wax, and a pigment component;
adding a first additive component to the emulsion, the first additive component including dimethicone and a PEG/PPG dimethicone compound;
adding medicament component to the emulsion including at least one of the following: bacitracin, neomycin, polymyxin, or pramoxine to form a medicated emulsion; and
adding a second additive component including bentonite to the medicated emulsion to form a homogenized medicated skin concealer.

15. The method of claim 14, further comprising:
preparing the aqueous component by combining a predominant amount of water with a minor amount of the magnesium aluminum silicate and the alkylene glycol at a first temperature;
preparing the oil component by combining a predominant amount of the plurality of different polyorganosiloxanes with a minor amount of the oil and the wax at a second temperature; and
adding the pigment component to the oil component at third temperature, the pigment component including a two or more of titanium dioxide, red iron oxide, yellow iron oxide, or black iron oxide.

16. The method of claim 15, wherein:
the alkylene glycol is propylene glycol;
the plurality of polyorganosiloxanes includes at least dimethicone, cyclomethicone, and C₃₀₋₄₅ alkyl dimethicone;
the oil is mineral oil;
the wax is carnauba wax; and
the first additive is dimethicone and PEG/PPG 18/18 dimethicone.

17. The method of claim 15, wherein:
the combining of the aqueous component and the oil component to form the emulsion occurs at a fourth temperature;
the adding of the first additive component to the emulsion occurs at a fifth temperature;
the adding of the medicament component to the emulsion occurs at a sixth temperature to form a medicated emulsion;
the adding of the second additive component to the medicated emulsion occurs at a seventh temperature;
the first, second, third, and fourth temperatures are independently greater than 50° C.; and
the seventh temperature is lower than the sixth temperature, the sixth temperature is lower than the fifth temperature, and the fifth temperature is lower than the first, second, third, and fourth temperatures.

18. A method of covering a wound in a skin of a patient, the method comprising:
applying an effective amount of a medicated skin concealer to a wound, the medicated skin concealer including a concealer composition and a medicament component, the medicament component including at least one of bacitracin, neomycin, polymyxin, or pramoxine.

19. The method of claim 18, wherein the concealer composition is an emulsion comprising:
an aqueous component including a predominant amount of water with a minor amount of a magnesium aluminum silicate and an alkylene glycol;
an oil component including a predominant amount of a plurality of different polyorganosiloxanes with a minor amount of an oil, a wax, and a pigment component;
a first additive component including dimethicone and a PEG/PPG dimethicone compound; and
a second additive component including bentonite.

20. The method of claim 18, wherein applying the effective amount of the medicated skin concealer includes applying about 0.1 to about 5 grams/cm² of a surface area of the wound every 2-12 hours.
